(19) United States
(12) Patent Application Publication
(10) Pub. No.: US 2019/0015045 A1
(43) Pub. Date: Jan. 17, 2019

(54) METHOD AND APPARATUS FOR DETECTING WEARING STATE OF A WEARABLE DEVICE

(71) Applicant: Goertek Inc., Weifang City, Shandong Province (CN)

(72) Inventor: Haibo LI, Weifang City (CN)

(21) Appl. No.: 16/068,617

(22) PCT Filed: Jan. 25, 2017
(86) PCT No.: PCT/CN2017/072542

§ 371 Date: Jul. 6, 2018

(30) Foreign Application Priority Data
Feb. 4, 2016 (CN) 201610079350.0

(51) Int. Cl. A61B 5/00; G01P 15/08; G04G 21/02; A61B 5/0205

(52) U.S. Cl. CPC A61B 5/681; G01P 15/0802; A61B 5/02438; A61B 5/02055; G04G 21/025

(57) ABSTRACT

A method for detecting a wearing state of a wearable device, comprising: detecting an acceleration of the wearable device by using an acceleration sensor and activating the detection of wearing state of the wearable device when the acceleration of the wearable device changes: detecting the distance between the wearable device and an adjacent object by using a proximity sensor, and activating a heart rate sensor and a temperature sensor when the distance between the wearable device and the adjacent object is less than a preset distance threshold; and determining that the wearable device is in a state of being worn when the data detected by the heart rate sensor satisfy a preset heart rate condition, and a surface temperature of the adjacent object detected by the temperature sensor satisfies a preset temperature condition. Also disclosed is an apparatus for detecting a wearing state of a wearable device.

### TECHNICAL FIELD

The present disclosure relates to the technical field of state identifying, and particularly relates to a method and apparatus for detecting a wearing state of a wearable device.

### BACKGROUND

As mobile technology continuously advances, the use of smart watches has been increasingly widespread as the next hotspot of the smart terminal industry. However, due to the limited volume of smart watches, the capacity of its battery cannot be very large, so its battery endurance capability is limited. Therefore, it is an important research direction to effectively reduce the electric power consumption.

Some functions in smart watches, such as step counting function and health data recording function, can provide effective or meaningful data feedback only when the user is wearing the smart watch. When the user is not wearing the smart watch, the activating of the above functions of the smart watch will cause the waste of electric power, and thus shorten the run time of the smart watch.

### SUMMARY

In view of the above analyses, the present disclosure provides a method and apparatus for detecting a wearing state of a wearable device, to solve the problem of wasting electric power due to the running of useless application programs when the wearable device is not worn.

To achieve the above objects, the present disclosure provides the following technical solutions:

In an aspect, the present disclosure provides a method for detecting a wearing state of a wearable device, the wearable device is provided therein with an acceleration sensor, a proximity sensor, a heart rate sensor and a temperature sensor, and the method comprises:
detecting an acceleration of the wearable device by using the acceleration sensor, and activating the detection of wearing state of the wearable device when the acceleration of the wearable device changes:
activating the proximity sensor to detect a distance between the wearable device and an adjacent object, activating the heart rate sensor and the temperature sensor when the distance between the wearable device and the adjacent object is less than a preset distance threshold, and determining that the wearable device is in a state of being worn when data detected by the heart rate sensor satisfy a preset heart rate condition, and a surface temperature of the adjacent object detected by the temperature sensor satisfies a preset temperature condition.

In another aspect, the present disclosure further provides an apparatus for detecting a wearing state of a wearable device, wherein the wearable device is provided therein with an acceleration sensor, a proximity sensor, a heart rate sensor and a temperature sensor, and the apparatus for detecting the wearing state comprises:
an activating unit, for detecting an acceleration of the wearable device by using the acceleration sensor, and activating the proximity sensor to detect the wearing state of the wearable device when the acceleration of the wearable device changes; and
a detecting unit, for detecting a distance between the wearable device and an adjacent object by using the proximity sensor, activating the heart rate sensor and the temperature sensor when the distance between the wearable device and the adjacent object is less than a preset distance threshold, and determining that the wearable device is in a state of being worn when the data detected by the heart rate sensor satisfy a preset heart rate condition, and a surface temperature of the adjacent object detected by the temperature sensor satisfies a preset temperature condition.

The advantageous effects of the embodiments of the present disclosure are as follows. Based on the fact that when the wearing state of the wearable device changes, the acceleration sensed by its acceleration sensor will changes, and the acceleration sensor has a lower power consumption than other sensors, the present disclosure uses the acceleration sensor to activate the wearing state detecting of the wearable device, to achieve the object of effectively reducing the electric power consumption of the wearable device; moreover, when the wearing state detecting is activated, the present disclosure comprehensively uses the proximity sensor, the heart rate sensor and the temperature sensor to accurately detect the wearing state of the wearable device.

In a preferable technical solution, the present disclosure periodically and cyclically detects the wearable device that is in a state of being worn, and when the wearable device switches to a state of not being worn, the wearing state detecting of the wearable device is turned off in time, thereby further reducing the electric power consumption of the wearable device.

### BRIEF DESCRIPTION OF DRAWINGS

FIG. 1 is a flow chart of a method for detecting a wearing state of a wearable device according to the first embodiment;

FIG. 2 is a perspective view of an inner surface of the back of a smart watch according to the first embodiment;

FIG. 3 is a schematic diagram of an outer surface of the back of a smart watch according to the first embodiment;

FIG. 4 is a flow chart of a method for detecting a wearing state of a smart watch according to the first embodiment;

FIG. 5 is a flow chart of using a timer to further judge the wearing state of the smart watch according to the first embodiment; and

FIG. 6 is a structural schematic diagram of an apparatus for detecting a wearing state of a wearable device according to the second embodiment.

### DETAILED DESCRIPTION

Regarding some application programs in smart watches, such as relevant application programs based on movement state detection or based on health state detection, only when the user is wearing the smart watch, the contents outputted by these application programs are meaningful. Therefore, the present disclosure, by detecting and identifying the wearing state of the smart watch, automatically turns on or turns off the relevant application programs according to the current wearing state of the smart watch, to reduce the electric power consumption of the smart watch and prolong the run time of the smart watch.

When the wearing state of the smart watch is detected and identified, the state identification technique and the sensor fusion technique will be used. The state identification technique is mainly to conduct comprehensive processing and analysis to obtain the state of the smart watch according to relevant environmental information gathered by the smart watch and relevant information of the smart watch itself such as the running state, the power supply state and the screen state. The sensor fusion is a relatively complicated technique, and it combines outputs of different sensors to obtain a more accurate identifying result when conducting state identifying.

The sensor fusion technique used in the present disclosure refers to the fusing of a sensor for movement detecting, a sensor for distance detecting, a sensor for heart rate detecting and a sensor for temperature detecting. The acceleration signal sensed by the acceleration sensor may be used to judge whether the smart watch is in the movement state, the distance signal sensed by the proximity sensor may be used to detect the distance between the wrist of the user and the smart watch in real time, the heart rate signal sensed by the heart rate sensor may be used to measure the heart rate of the user wearing the smart watch, and the temperature sensor may be used to detect the body surface temperature of the user wearing the smart watch.

The inventive concept of the present disclosure is as follows. Based on the fact that when the wearing state of the wearable device changes, the acceleration sensed by its acceleration sensor will changes, and the acceleration sensor has a lower power consumption than other sensors, the present disclosure uses the acceleration sensor to activate the wearing state detecting of the wearable device, and when the wearing state detecting is activated, the proximity sensor, the heart rate sensor and the temperature sensor are comprehensively used to detect the wearing state of the wearable device.

In order to make the objects, the technical solutions and the advantages of the present disclosure clearer, the embodiments of the present disclosure will be further described below in detail in conjunction with the drawings.

### First Embodiment

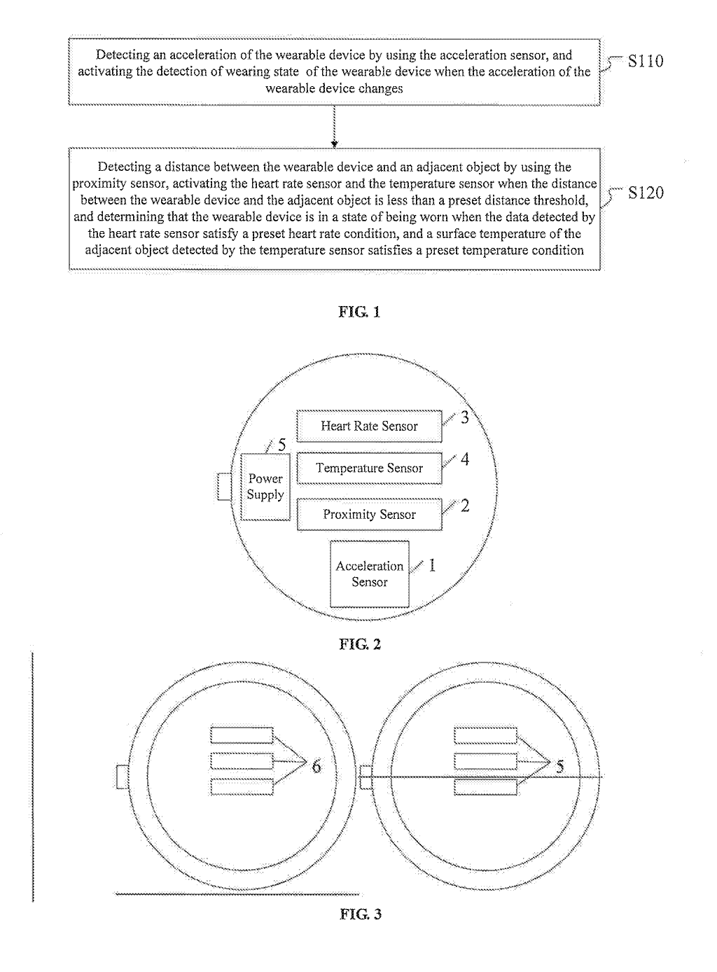

FIG. 1 is a flow chart of a method for detecting the wearing state of a wearable device according to the present embodiment. The wearable device is provided therein with an acceleration sensor, a proximity sensor, a heart rate sensor and a temperature sensor. As shown in FIG. 1, the method comprises:

S110, detecting an acceleration of the wearable device by using the acceleration sensor, and activating the detection of wearing state of the wearable device when the acceleration of the wearable device changes:

S120, detecting a distance between the wearable device and an adjacent object by using the proximity sensor, activating the heart rate sensor and the temperature sensor when the distance between the wearable device and the adjacent object is less than a preset distance threshold, and determining that the wearable device is in a state of being worn when the data detected by the heart rate sensor satisfy a preset heart rate condition, and a surface temperature of the adjacent object detected by the temperature sensor satisfies a preset temperature condition.

In Step S120, comprehensively considering the requirement on the accuracy of the heart rate data detected by the heart rate sensor and the requirement on the effective utilization of the electric power consumption of the wearable device, in the present embodiment, preferably, the detection duration of the heart rate sensor is set to be 5 to 15 seconds. It is because, the heart rate sensor in the working state will consume much energy, if the detection duration is too long, a large amount of electrical energy will be consumed, but if the detection duration is too short, the data detected by the heart rate sensor may not be accurate.

It should be noted that, the adjacent object in the present embodiment refers to the nearest object facing the wearing surface of the wearable device. The smart watch is particularly described as an example. The surface of the smart watch that contacts with the wrist of the user is its wearing surface. When the smart watch is worn on the wrist of the user, even if the front face of the smart watch is close to or contacts with other objects, at this point the adjacent object refers to the wrist of the user. When the smart watch is placed on a table surface and the wearing surface of the smart watch contacts with the table surface, even if the front face of the smart watch is close to or contacts with other objects, at this point the adjacent object still refers to the wrist of the user.

A particular implementation of the wearing state detecting of the wearable device in Step S120 of the present embodiment is:
acquiring a distance between the wearable device and an adjacent object detected by the proximity sensor, and when the distance between the wearable device and the adjacent object is less than 5 mm, activating the heart rate sensor and the temperature sensor; and
acquiring detected data of the heart rate sensor in a preset duration and a surface temperature of the adjacent object detected by the temperature sensor, and when the detected data are between 40 and 220 times/minute and the surface temperature is between 34 and 42 degrees Celsius, determining that the wearable device is in a state of being worn; wherein the preset duration is preferably 5 to 15 seconds.

The method in FIG. 1, after determining that the wearable device is in a state of being worn, further comprises:
judging whether the wearable device is still in a state of being worn at a preset time interval, and when the wearable device is in a state of not being worn, turning off the wearing state detecting of the wearable device, and judging whether the acceleration of the wearable device changes.

It should be noted that, “turning off the wearing state detecting of the wearable device” mentioned in the present embodiment should be understood as turning off relevant hardware entities and functional components involved in the processing of the above Step S120, to stop performing the function of wearing state detecting of the wearable device, such as turning off relevant hardware entities such as the proximity sensor, the heart rate sensor and the temperature sensor, and/or turning off the logic unit for determining and identifying.

The step of judging whether the wearable device is still in a state of being worn at a preset time interval comprises:
detecting a distance between the wearable device and a human body by using the proximity sensor, and when the distance between the wearable device and the human body is less than a preset distance threshold, judging whether the wearable device is still in a state of being worn according to the heart rate in a preset duration detected by the heart rate sensor and/or according to the body surface temperature detected by the temperature sensor.

In the present embodiment, preferably, the preset distance threshold is 5 mm, the preset heart rate condition is 40˜220 times/minute, and the preset temperature condition is 34˜42 degrees Celsius.

It should be noted that, in the present disclosure, regarding the wearable devices that need to be taken off the body of the user to charge, namely, the wearable devices that need to be charged in a state of not being worn, when it is detected that the wearable device is in a charging state, the wearing state detecting of the wearable device is turned off.

Particularly, the method in FIG. 1 further comprises: detecting a power supply state of the wearable device in real time, and if the wearable device is in a charging state, turning off the wearing state detecting of the wearable device.

It should be further noted that, in the present embodiment, in order to improve the accuracy of the wearing state detecting of the wearable device, preferably, the proximity sensor, the heart rate sensor and the temperature sensor are provided at the positions of the wearable device that contact with the body of the user.

The present embodiment, by using the acceleration sensor provided in the wearable device, detects the acceleration of the wearable device according to the acceleration signal sensed by the acceleration sensor, and when the acceleration of the wearable device changes, activates wearing state detecting of the wearable device; when the wearing state detecting of the wearable device is activated, comprehensively uses the proximity sensor, the heart rate sensor and the temperature sensor; and only when the distance between the wearable device and the adjacent object satisfies a preset condition, activates the heart rate sensor and the temperature sensor to determine the wearing state of the wearable device. In other words, the present embodiment uses the acceleration sensor having a relatively low power consumption to trigger the wearing state detecting of the wearable device, to achieve the object of effectively reducing the electric power consumption of the wearable device. Furthermore, when the wearable device is in a state of being worn, the present embodiment periodically and cyclically detects the state of the wearable device, and can turn off in time the function of wearing state detecting of the wearable device when the wearable device switches to a state of not being worn, thereby further reducing the electric power consumption of the wearable device.

In a particular embodiment, the wearable device is a smart watch. As shown in FIG. 2, the smart watch has an acceleration sensor 1, a proximity sensor 2, a heart rate sensor 3, a temperature sensor 4 and a power supply 5.

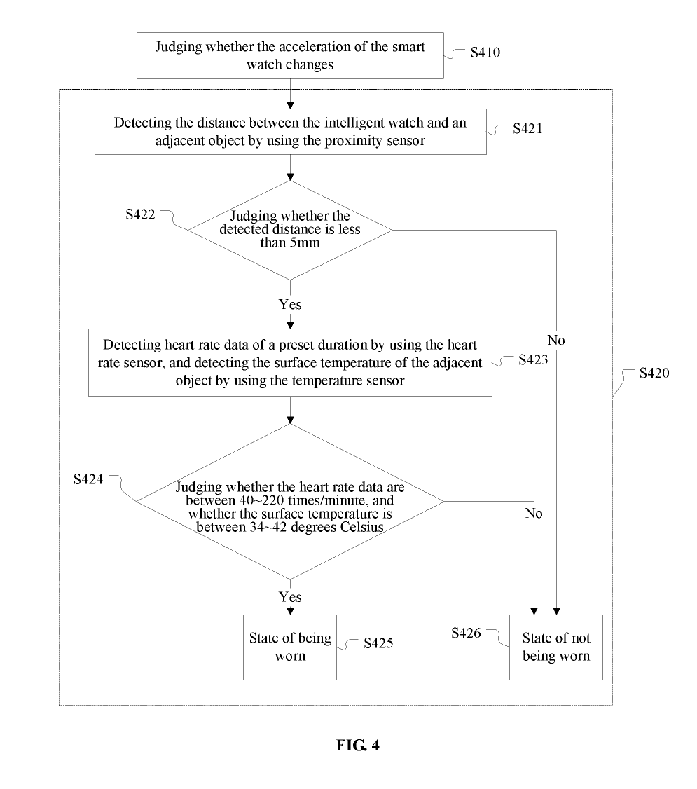

As shown in FIG. 3, the proximity sensor 2, the heart rate sensor 3 and the temperature sensor 4 are provided at the back of the smart watch, namely, the side of the smart watch that contacts with the wrist of the user. Openings 6 are provided on an inner side surface of a housing of the smart watch that is corresponding to the proximity sensor 2, the heart rate sensor 3 and the temperature sensor 4, to facilitate detecting the external environment using the proximity sensor 2, the heart rate sensor 3 and the temperature sensor 4.

For illustration purposes, in the present particular embodiment, the preset distance threshold is 5 mm, the preset heart rate condition is 40˜220 times/minute, and the preset temperature condition is 34˜42 degrees Celsius.

FIG. 4 is a flow chart of a method for detecting the wearing state of a smart watch according to the present particular embodiment. As shown in FIG. 4, the wearing state of the smart watch is detected by the following method:

S410, judging whether the acceleration of the smart watch changes according to the acceleration signal that is sensed by the acceleration sensor of the smart watch, and when it is determined that the acceleration of the smart watch changes, executing Step S420.

In this step, when it is determined that the acceleration of the smart watch does not changes, it is chosen to judge whether the acceleration of the smart watch changes at the next moment according to the application demands.

S420, activating the wearing state detecting of the smart watch.

The particular flow process of activating the wearing state detecting of the smart watch in Step S420 is as follows:

S421, activating the proximity sensor, and detecting the distance between the smart watch and an adjacent object by using the proximity sensor.

S422, judging whether the distance between the smart watch and the adjacent object detected by the proximity sensor is less than 5 mm, and when the distance between the smart watch and the adjacent object detected by the proximity sensor is less than 5 mm, executing Step S423, and if no, executing Step S426.

S423, activating the heart rate sensor and the temperature sensor, detecting heart rate data of a preset duration by using the heart rate sensor, and detecting the surface temperature of the adjacent object by using the temperature sensor.

S424, judging whether the heart rate data detected by the heart rate sensor are between 40˜220 times/minute, and whether the surface temperature detected by the temperature sensor is between 34˜42 degrees Celsius, and when the detected heart rate data are between 40˜220 times/minute and the detected surface temperature is between 34˜42 degrees Celsius, executing Step S425, and if no, executing Step S426.

S425, determining that the smart watch is in a state of being worn.

S426, determining that the smart watch is in a state of not being worn.

In practical usage scenarios, the smart watch may switch from a state of being worn to a state of not being worn. For example, when the user is having a rest, the smart watch is often taken off. At this point, relevant application programs based on movement state detection or based on health state detection should be turned off, to achieve the object of reducing the electric power consumption of the smart watch.

Therefore, the present particular embodiment, after executing Step S440, judges whether the smart watch is still in a state of being worn at a preset time interval, for example every 30 minutes, and when the smart watch is in a state of not being worn, turns off the wearing state detecting of the smart watch, and returns to Step S410 to judge whether the acceleration of the smart watch changes.

In an implementation of the present particular embodiment, when it is determined that the smart watch is in a state of being worn, the present disclosure may activate a timer, and further judge the wearing state of the smart watch when a timeout occurs in the timer.

As shown in FIG. 5, the flow process of using the timer to further judge the wearing state of the smart watch is as follows:

S510, when it is determined that the smart watch is in a state of being worn, activating a timer, and when a timeout occurs in the timer, executing Step S520.

S520, judging whether the smart watch switches from a state of being worn to a state of not being worn.

The flow process of detecting the change of the wearing state of the smart watch in Step S520 is as follows:

S521, detecting the distance between the smart watch and a wrist by using the proximity sensor.

S522, judging whether the distance between the smart watch and the wrist detected by the proximity sensor is less than 5 mm, and when the distance between the smart watch and the wrist detected by the proximity sensor is less than 5 mm, executing Step S523, and if no, executing Step S526.

S523, detecting heart rate data in a preset duration by using the heart rate sensor, and detecting the body surface temperature of the wrist by using the temperature sensor.

S524, judging whether the heart rate data detected by the heart rate sensor are between 40˜220 times/minute, and whether the body surface temperature detected by the temperature sensor is between 34˜42 degrees Celsius, and when the detected heart rate data are between 40˜220 times/minute and the detected body surface temperature is between 34˜42 degrees Celsius, executing Step S525, and if no, executing Step S526.

S525, determining that the smart watch is still in a state of being worn, and returning to Step S510.

S526, determining that the smart watch has switched to a state of not being worn, turning off the wearing state detecting of the smart watch, and going to Step S410 in FIG. 4.

It should be noted that, when a timeout occurs in the timer, the wearing state of the smart watch may be judged by using the above method in Steps S510˜S520, or by merely using the proximity sensor and the temperature sensor, or by merely using the proximity sensor and the heart rate sensor. It is because, at this point, the wearing state of the smart watch is determined merely in order to judge whether the smart watch has switched from a state of being worn to a state of not being worn, so an accurate result can be obtained by merely using the proximity sensor and the temperature sensor or by merely using the proximity sensor and the heart rate sensor when the wearing state of the smart watch is detected.

It should be further noted that, at present most smart watches must be taken off the wrist of the user when being charged, so in the present particular embodiment, the wearing state detecting of the smart watch is turned off immediately when it is detected that the power supply 5 is in a charging state. Only when the power supply state of the smart watch is in a non-charging state, the above flow process is executed.

### Second Embodiment

Based on the same technical concept as that of the first embodiment, the present embodiment provides an apparatus for detecting the wearing state of a wearable device. The wearable device is provided therein with an acceleration sensor, a proximity sensor, a heart rate sensor and a temperature sensor.

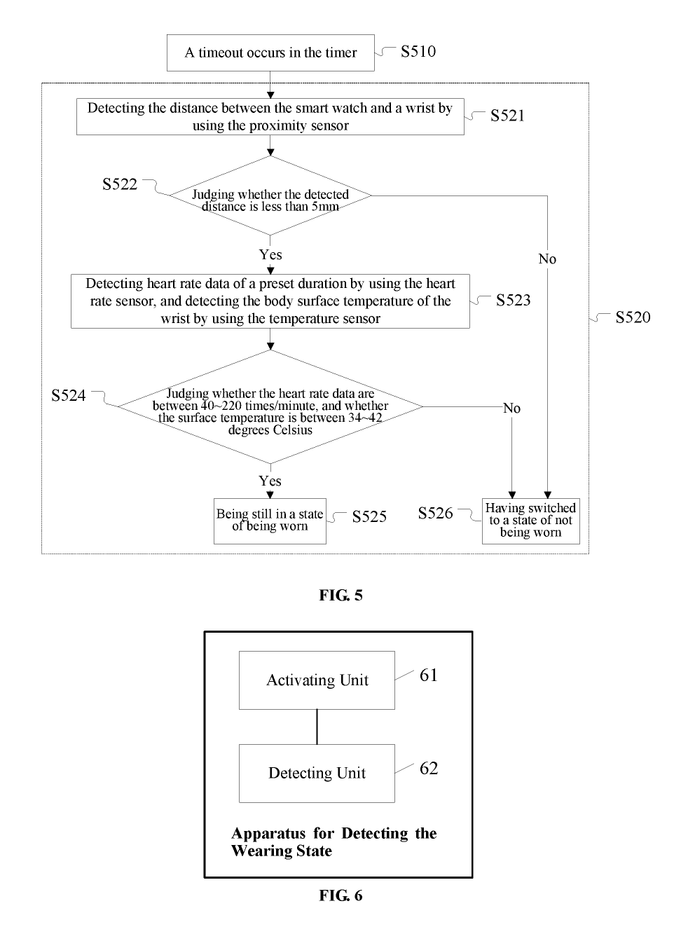

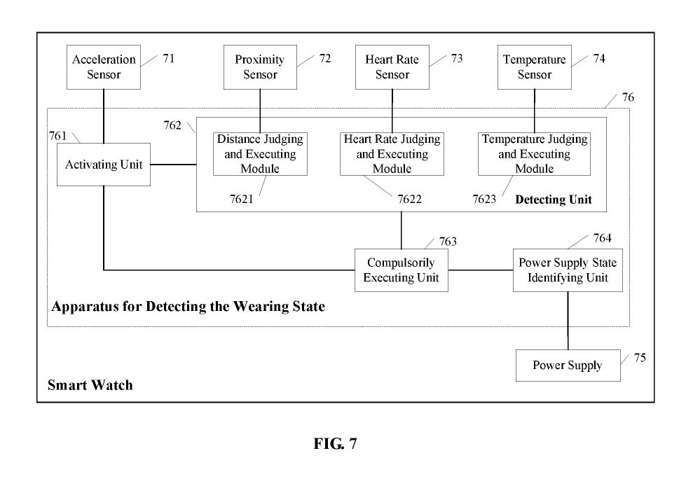

FIG. 6 is a structural schematic diagram of an apparatus for detecting the wearing state of a wearable device according to the second embodiment. As shown in FIG. 6, the apparatus for detecting the wearing state in FIG. 6 comprises:
an activating unit 61, for detecting an acceleration of the wearable device by using the acceleration sensor, and activating the proximity sensor to detect the wearing state of the wearable device when the acceleration of the wearable device changes; and
a detecting unit 62, for detecting a distance between the wearable device and an adjacent object by using the proximity sensor, activating the heart rate sensor and the temperature sensor when the distance between the wearable device and the adjacent object is less than a preset distance threshold, and w determining that the wearable device is in a state of being worn when the data detected by the heart rate sensor satisfy a preset heart rate condition, and a surface temperature of the adjacent object detected by the temperature sensor satisfies a preset temperature condition.

The detecting unit 62 comprises:
a first acquiring module, for acquiring a distance between the wearable device and an adjacent object detected by the proximity sensor;
a first judging and processing module, for activating the heart rate sensor and the temperature sensor when the distance between the wearable device and the adjacent object acquired by the first acquiring module is less than 5 mm;
a second acquiring module, for acquiring detected data of the heart rate sensor in a preset duration and a surface temperature of the adjacent object detected by the temperature sensor; and
a second judging and processing module, for determining that the wearable device is in a state of being worn when the detected data acquired by the second acquiring module are between 40 and 220 times/minute and the surface temperature acquired by the second acquiring module is between 34 and 42 degrees Celsius; wherein the preset duration is preferably 5 to 15 seconds.

Preferably, the apparatus for detecting the wearing state in FIG. 6 further comprises a compulsorily executing unit.

The detecting unit 62 is further for judging whether the wearable device is still in a state of being worn at a preset time interval. Particularly, the detecting unit 62 is for detecting a distance between the wearable device and a human body by using the proximity sensor, and when the distance between the wearable device and the human body is less than a preset distance threshold, judging whether the wearable device is still in a state of being worn according to the heart rate detected by the heart rate sensor and/or according to the body surface temperature detected by the temperature sensor.

The compulsorily executing unit is for turning off the detecting unit 62 when the detecting unit 62 detects that the wearable device is in a state of not being worn, and driving the starting-up unit 61 to judge whether the acceleration of the wearable device changes.

In the present embodiment, preferably, the preset distance threshold is 5 mm, the preset heart rate condition is 40˜220 times/minute, and the preset temperature condition is 34˜42 degrees Celsius.

It should be noted that, in the present disclosure, regarding the wearable devices that need to be taken off the body of the user to charge, namely, the wearable devices that need to be charged in a state of not being worn, when it is detected that the wearable device is in a charging state, the wearing state detecting of the wearable device is turned off.

Particularly, the apparatus for detecting the wearing state in FIG. 6 further comprises: a power supply state identifying unit, for detecting the power supply state of the wearable device; and
the compulsorily executing unit is further for turning off the detecting unit when the power supply state identifying unit detects that the wearable device is in a charging state.

It should be noted that, in the present embodiment, in order to improve the accuracy of the wearing state detecting of the wearable device, preferably, the proximity sensor, the heart rate sensor and the temperature sensor are provided at the positions of the wearable device that contact with the body of the user.

In a particular embodiment, the wearable device is a smart watch. A proximity sensor, a heart rate sensor and a temperature sensor of the smart watch are provided at the back of the smart watch, namely, the side of the smart watch that contacts with the wrist of the user. Openings are provided on an inner side surface of a housing of the smart watch that is corresponding to the proximity sensor, the heart rate sensor and the temperature sensor, to facilitate detecting the external environment using the proximity sensor, the heart rate sensor and the temperature sensor.

For illustration purposes, in the present particular embodiment, the preset distance threshold is 5 mm, the preset heart rate condition is 40˜220 times/minute, and the preset temperature condition is 34˜42 degrees Celsius.

FIG. 7 is a structural schematic diagram of a smart watch of the present embodiment. As shown in FIG. 7, the smart watch comprises: an acceleration sensor 71, a proximity sensor 72, a heart rate sensor 73, a temperature sensor 74, a power supply 75 and an apparatus for detecting the wearing state 76. The apparatus for detecting the wearing state 76 comprises: an activating unit 761, a detecting unit 762, a compulsorily executing unit 763 and a power supply state identifying unit 764.

The detecting unit 762 comprises: a distance judging and executing module 7621, a heart rate judging and executing module 7622 and a temperature judging and executing module 7623.

In the present particular embodiment, the working process of the apparatus for detecting the wearing state of the smart watch is as follows.

The acceleration sensor 71 sends the acceleration signal that it senses to the activating unit 761. The activating unit 761 calculates the current acceleration of the smart watch according to the received acceleration signal, and drives the detecting unit 762 to activate the wearing state detecting of the smart watch when it is determined that the acceleration of the smart watch changes.

Particularly, the activating unit 761 activates the proximity sensor 72 to enable the proximity sensor 72 to detect the distance between the smart watch and an adjacent object, and sends the detected distance signal to the distance judging and executing module 7621. The distance judging and executing module 7621 judges whether the received distance signal is less than 5 mm, and when the received distance signal is less than 5 mm, activates the heart rate sensor 73 and the temperature sensor 74, to enable the heart rate sensor 73 to send the detected heart rate signal in a preset duration to the heart rate judging and executing module 7622, and to enable the temperature sensor 74 to send the detected surface temperature signal of the adjacent object to the temperature judging and executing module 7623. The heart rate judging and executing module 7622 judges whether the heart rate value corresponding to the heart rate signal detected by the heart rate sensor 73 is between 40˜220 times/minute, and the temperature judging and executing module 7623 judges whether the temperature value corresponding to the surface temperature signal detected by the temperature sensor 74 is between 34˜42 degrees Celsius. When the detected heart rate value corresponding to the heart rate signal is between 40˜220 times/minute, and the detected temperature value corresponding to the surface temperature signal is between 34˜42 degrees Celsius, the detecting unit 762 determines that the smart watch is in a state of being worn, and if no, the detecting unit 762 determines that the smart watch is in a state of not being worn.

In practical usage scenarios, the smart watch may switch from a state of being worn to a state of not being worn. For example, when the user is having a rest, the smart watch is often taken off. At this point, relevant application programs based on movement state detection or based on health state detection should be turned off, to achieve the object of reducing the electric power consumption of the smart watch.

In the present particular embodiment, the detecting unit 762 judges whether the smart watch is still in a state of being worn at a preset time interval, for example every 30 minutes, and when the smart watch is in a state of not being worn, the compulsorily executing unit 763 turns off the detecting unit 762 of the smart watch, and drives the acceleration sensor 71 to detect the movement state of the smart watch.

It should be noted that, at present most smart watches must be taken off the wrist of the user when being charged, so in the present particular embodiment, when the power supply state identifying unit 764 detects that the power supply 75 is in a charging state, the compulsorily executing unit 763 turns off the detecting unit 762 of the smart watch. Only when the state of the power supply 75 of the smart watch is in a non-charging state, the above flow process is executed.

In conclusion, the present disclosure discloses a method and apparatus for detecting the wearing state of a wearable device. Based on the fact that when the wearing state of the wearable device changes, the acceleration sensed by its acceleration sensor will changes, and the acceleration sensor has a lower power consumption than other sensors, the present disclosure uses the acceleration sensor to activate the wearing state detecting of the wearable device, to achieve the object of effectively reducing the electric power consumption of the wearable device; moreover, when the wearing state detecting is activated, the present disclosure comprehensively uses the proximity sensor, the heart rate sensor and the temperature sensor to accurately detect the wearing state of the wearable device. In a preferable technical solution, the present disclosure periodically and cyclically detects the wearable device that is in a state of being worn, and when the wearable device switches to a state of not being worn, the wearing state detecting of the wearable device is turned off in time, thereby further reducing the electric power consumption of the wearable device.

The above description is merely preferable embodiments of the present disclosure, and is not intended to limit the protection scope of the present disclosure. Any modifications, equivalent substitutions or improvements made within the spirit and principle of the present disclosure shall all be included in the protection scope of the present disclosure.

1. A method for detecting a wearing state of a wearable device, wherein the wearable device is provided therein with an acceleration sensor, a proximity sensor, a heart rate sensor and a temperature sensor, and the method comprises:
detecting an acceleration of the wearable device by using the acceleration sensor, and activating the detection of wearing state of the wearable device when the acceleration of the wearable device changes:
activating the proximity sensor to detect a distance between the wearable device and an adjacent object, activating the heart rate sensor and the temperature sensor when the distance between the wearable device and the adjacent object is less than a preset distance threshold, and determining that the wearable device is in a state of being worn when data detected by the heart rate sensor satisfy a preset heart rate condition, and a surface temperature of the adjacent object detected by the temperature sensor satisfies a preset temperature condition.

2. The method for detecting a wearing state according to claim 1, wherein activating the detection of wearing state of the wearable device is particularly comprises:
acquiring a distance between the wearable device and an adjacent object detected by the proximity sensor, and when the distance between the wearable device and the adjacent object is less than 5 mm, activating the heart rate sensor and the temperature sensor; and
acquiring detected data of the heart rate sensor in a preset duration and a surface temperature of the adjacent object detected by the temperature sensor, and when the detected data are between 40 and 220 times/minute and the surface temperature is between 34 and 42 degrees Celsius, determining that the wearable device is in a state of being worn; wherein the preset duration is 5 to 15 seconds.

3. The method for detecting a wearing state according to claim 1, wherein after determining that the wearable device is in a state of being worn, the method further comprises:
judging whether the wearable device is still in a state of being worn at a preset time interval, and when the wearable device is in a state of not being worn, turning off the wearing state detecting of the wearable device, and judging whether the acceleration of the wearable device changes.

4. The method for detecting a wearing state according to claim 3, wherein the step of judging whether the wearable device is still in a state of being worn at a preset time interval comprises:
detecting a distance between the wearable device and a human body by using the proximity sensor, and when the distance between the wearable device and the human body is less than the preset distance threshold, judging whether the wearable device is still in a state of being worn according to the heart rate detected by the heart rate sensor and/or according to the body surface temperature detected by the temperature sensor.

5. The method for detecting a wearing state according to claim 1, wherein the method further comprises:
detecting a power supply state of the wearable device, and if the wearable device is in a charging state, turning off the wearing state detecting of the wearable device.

6. The method for detecting the wearing state according to claim 2, wherein the method further comprises:
detecting a power supply state of the wearable device, and if the wearable device is in a charging state, turning off the wearing state detecting of the wearable device.

7. The method for detecting a wearing state according to claim 3, wherein the method further comprises:
detecting a power supply state of the wearable device, and if the wearable device is in a charging state, turning off the wearing state detecting of the wearable device.

8. The method for detecting a wearing state according to claim 4, wherein the method further comprises:
detecting a power supply state of the wearable device, and if the wearable device is in a charging state, turning off the wearing state detecting of the wearable device.

9. The method for detecting a wearing state according to claim 5, wherein the wearable device is a smart watch, and the proximity sensor, the heart rate sensor and the temperature sensor are provided at a side of the smart watch that contacts with a wrist of the user; and
openings are provided on an inner side surface of a housing of the smart watch that is corresponding to the proximity sensor, the heart rate sensor and the temperature sensor, to facilitate detecting an external environment using the proximity sensor, the heart rate sensor and the temperature sensor.

10. The method for detecting a wearing state according to claim 6, wherein the wearable device is a smart watch, and the proximity sensor, the heart rate sensor and the temperature sensor are provided at a side of the smart watch that contacts with a wrist of the user; and
openings are provided on an inner side surface of a housing of the smart watch that is corresponding to the proximity sensor, the heart rate sensor and the temperature sensor, to facilitate detecting an external environment using the proximity sensor, the heart rate sensor and the temperature sensor.

11. The method for detecting a wearing state according to claim 7, wherein the wearable device is a smart watch, and the proximity sensor, the heart rate sensor and the temperature sensor are provided at a side of the smart watch that contacts with a wrist of the user; and
openings are provided on an inner side surface of a housing of the smart watch that is corresponding to the proximity sensor, the heart rate sensor and the temperature sensor, to facilitate detecting an external environment using the proximity sensor, the heart rate sensor and the temperature sensor.

12. The method for detecting a wearing state according to claim 8, wherein the wearable device is a smart watch, and the proximity sensor, the heart rate sensor and the temperature sensor are provided at a side of the smart watch that contacts with a wrist of the user; and
openings are provided on an inner side surface of a housing of the smart watch that is corresponding to the proximity sensor, the heart rate sensor and the temperature sensor, to facilitate detecting an external environment using the proximity sensor, the heart rate sensor and the temperature sensor.

13. An apparatus for detecting a wearing state of a wearable device, wherein the wearable device is provided therein with an acceleration sensor, a proximity sensor, a heart rate sensor and a temperature sensor, and the apparatus for detecting the wearing state comprises:
an activating unit, for detecting an acceleration of the wearable device by using the acceleration sensor, and activating a detecting unit to detect the wearing state of the wearable device when the acceleration of the wearable device changes; and
the detecting unit, for detecting a distance between the wearable device and an adjacent object by using the proximity sensor, activating the heart rate sensor and the temperature sensor when the distance between the wearable device and the adjacent object is less than a preset distance threshold, and determining that the wearable device is in a state of being worn when the data detected by the heart rate sensor satisfy a preset heart rate condition, and a surface temperature of the adjacent object detected by the temperature sensor satisfies a preset temperature condition.

14. The apparatus for detecting a wearing state according to claim 13, wherein the detecting unit comprises:
a first acquiring module, for acquiring a distance between the wearable device and an adjacent object detected by the proximity sensor;
a first judging and processing module, for activating the heart rate sensor and the temperature sensor when the distance between the wearable device and the adjacent object acquired by the first acquiring module is less than 5 mm;
a second acquiring module, for acquiring detected data of the heart rate sensor in a preset duration and a surface temperature of the adjacent object detected by the temperature sensor; and
a second judging and processing module, for determining that the wearable device is in a state of being worn when the detected data acquired by the second acquiring module are between 40 and 220 times/minute and the surface temperature acquired by the second acquiring module is between 34 and 42 degrees Celsius; wherein the preset duration is 5 to 15 seconds.

15. The apparatus for detecting a wearing state according to claim 13, wherein the apparatus for detecting the wearing state further comprises a compulsorily executing unit;
the detecting unit is further for judging whether the wearable device is still in a state of being worn at a preset time interval; and
the compulsorily executing unit is for turning off the detecting unit when the detecting unit detects that the wearable device is in a state of not being worn, and driving the activating unit to judge whether the acceleration of the wearable device changes.

16. The apparatus for detecting a wearing state according to claim 14, wherein the apparatus for detecting the wearing state further comprises a compulsorily executing unit;
the detecting unit is further for judging whether the wearable device is still in a state of being worn at a preset time interval; and
the compulsorily executing unit is for, turning off the detecting unit when the detecting unit detects that the wearable device is in a state of not being worn, and driving the activating unit to judge whether the acceleration of the wearable device changes.

17. The apparatus for detecting a wearing state according to claim 15, wherein the apparatus for detecting the wearing state further comprises a power supply state identifying unit;
the power supply state identifying unit is for detecting a power supply state of the wearable device; and
the compulsorily executing unit is for, turning off the detecting unit when the power supply state identifying unit detects that the wearable device is in a charging state.

18. The apparatus for detecting a wearing state according to claim 16, wherein the apparatus for detecting the wearing state further comprises a power supply state identifying unit;
the power supply state identifying unit is for detecting a power supply state of the wearable device; and
the compulsorily executing unit is for, turning off the detecting unit when the power supply state identifying unit detects that the wearable device is in a charging state.
